(19) United States
(12) Patent Application Publication
(10) Pub. No.: US 2025/0017455 A1
(43) Pub. Date: Jan. 16, 2025

(54) ENDOSCOPE WITH PERSPECTIVE VIEW

(71) Applicant: Covidien LP, Mansfield, MA (US)

(72) Inventors: Shai Tofach, Louisville, CO (US); Peter Douglas Colin Inglis, Boulder, CO (US); Derek S. Tata, Longmont, CO (US)

(21) Appl. No.: 18/753,380

(22) Filed: Jun. 25, 2024

Related U.S. Application Data
(60) Provisional application No. 63/513,178, filed on Jul. 12, 2023.

(51) Int. Cl. A61B 1/045; A61B 1/00; A61B 1/005; A61B 1/008; A61B 1/267

(52) U.S. Cl. CPC A61B 1/045; A61B 1/00006; A61B 1/0004; A61B 1/0005; A61B 1/0051; A61B 1/008; A61B 1/267

(57) ABSTRACT

The technology relates to systems and methods for an endoscope with a collapsible working channel and implementing a virtual anchor in an endoscope with a steerable tip. An example endoscope includes a proximal end, a steerable distal tip including an articulating segment, and a collapsible working channel, positioned on an outer surface of the endoscope, capable of selectively collapsing and expanding to receive a surgical tool. The collapsible working channel includes a proximal opening and a distal opening positioned proximally from the articulating segment. Virtual anchoring may include setting a reference image frame, identifying a landmark feature, determining a first position of the landmark feature, receiving an updated image frame; identifying the landmark feature in the updated image frame, determining a second position of the landmark feature, and based on a difference between the first position and the second position: generating corrective steering signals or shifting a cropped region.

### CROSS-REFERENCE TO RELATED APPLICATION

The present application claims the benefit of U.S. Provisional Patent Application Ser. No. 63/513,178, filed on Jul. 12, 2023, the entire content of which is incorporated herein by reference.

### BACKGROUND

An endoscope is a narrow flexible tube that includes a camera and light source integrated into a steerable distal tip, which is inserted into a patient's body and used to view tissue and anatomical structures of the patient. The steerable tip is controlled by a clinician using a control device connected to the proximal end of the endoscope, which remains outside the body. The clinician may view images from the endoscope on a display associated with the control device. The endoscope may also include a working channel that may be used to pass a medical instrument through the interior of the endoscope and out of the distal tip, where the instrument may be used to perform a variety of procedures.

It is with respect to this general technical environment that aspects of the present technology disclosed herein have been contemplated. Furthermore, although a general environment is discussed, it should be understood that the examples described herein should not be limited to the general environment identified herein.

### SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter. Additional aspects, features, and/or advantages of examples will be set forth in part in the description which follows and, in part, will be apparent from the description, or may be learned by practice of the disclosure.

In an aspect, the technology relates to a method, performed by an endoscope controller, for controlling an endoscope. The method included receiving an image frame from a camera on a distal tip of an endoscope; receiving an indication to activate a virtual anchor; based on receiving the indication to activate the virtual anchor, setting the image frame as a reference image frame; identifying a landmark feature in the reference image frame; determining a first position of the landmark feature in the reference image frame; receiving an updated image frame from the camera; identifying the landmark feature in the updated image frame; determining a second position of the landmark feature in the reference image frame; and based on a difference between the first position and the second position, performing at least one of: generating corrective steering signals to steer the distal tip; or shifting a cropped region of the updated image frame.

In an example, the indication to activate the virtual anchor is a manual input received by the endoscope controller. In another example, the method further includes displaying the reference frame on a display of the endoscope controller, wherein identifying the landmark feature is based on manual input received via touch input received via the display. In still another example, the method further includes determining a secondary characteristic for the landmark feature in the reference frame; and determining the secondary characteristic of the landmark feature in the updated image frame, wherein determining the positions of the landmark feature is based on the secondary characteristic. A further example, the secondary characteristic is at least one of a centroid, a border, an area, a major axis, or a minor axis. In still yet another example, a first cropped region is set for the reference frame and shifting the cropped region of the updated image frame comprises shifting the first cropped region to a second cropped region that is in a different position than the first cropped region. In yet a further example, the landmark feature is identified in the updated image frame in an area outside of the first cropped region.

In another aspect, the technology relates to an endoscopic imaging system that includes an endoscope having a steerable distal tip including a camera; and an endoscope controller connected to the endoscope, the endoscope controller comprising a processor and memory, storing instructions that when executed by the processor, cause the endoscopic controller to perform operations. The operations include receive an indication to activate a virtual anchor; based on receiving the indication to activate the virtual anchor, set a current image frame from the camera as a reference image frame; identify a first position of a landmark feature in the reference image frame; receive an updated image frame captured subsequent to the reference image frame; identify a second position of the landmark feature in the updated image frame; and based on a difference between the first position and the second position, perform at least one of: generate corrective steering signals to steer the distal tip; or shift a cropped region of the updated image frame.

In an example, the endoscopic controller is a video laryngoscope. In another example, the corrective steering signals are generated to steer the distal tip. In still another example, the cropped region of the updated image frame is shifted. In yet another example, the endoscope further includes a collapsible working channel, positioned on an outer surface of the endoscope, capable of selectively collapsing and expanding to receive a surgical tool. The collapsible working channel includes a proximal opening; and a distal opening positioned proximally from steerable distal tip.

In another aspect, the technology relates to an endoscope that includes a proximal end; a steerable distal tip including an articulating segment; a collapsible working channel, positioned on an outer surface of the endoscope, capable of selectively collapsing and expanding to receive a surgical tool. The collapsible working channel includes a proximal opening; and a distal opening positioned proximally from the articulating segment.

In an example, the distal opening is offset from the articulating segment by a distance between 1-5 cm. In a further example, the articulating segment is a first articulating segment, and the endoscope further comprises a second articulating segment adjacent the first articulating segment, wherein the distal opening is positioned proximally from both the first and second articulating segments.

### BRIEF DESCRIPTION OF THE DRAWINGS

The following drawing figures, which form a part of this application, are illustrative of aspects of systems and methods described below and are not meant to limit the scope of the disclosure in any manner, which scope shall be based on the claims.

FIGS. 1A-1B depict an example video system that includes a steerable endoscope with a collapsible working channel.

FIG. 1C is a block diagram of another example video system.

FIG. 2A-B depict a front view of the distal tip of an example endoscope with a collapsible working channel.

FIG. 3A depicts a view of an example endoscope positioned within an anatomical lumen.

FIG. 3B-C depict the field of view of the example endoscope of FIG. 3A.

FIG. 4 depicts a side perspective view of an example endoscope and surgical tool.

FIG. 5 depicts an example method for implementing a virtual anchor.

FIG. 6 depicts another example method for implementing a virtual anchor.

### DETAILED DESCRIPTION

A medical endoscope is a narrow, flexible tube that includes a video camera system integrated into a steerable distal tip that is inserted into the patient's body. The proximal end of the endoscope remains outside of the body and is connected to a control device that allows a clinician to steer the distal tip and view video images acquired by the camera system. The endoscope may be navigated into an anatomical lumen or other cavity of a patient, such as the patient's airway, gastrointestinal (GI) tract, or another cavity. When navigated to a location of interest, the clinician may use the control device to articulate the endoscope steerable tip to establish a viewpoint of an anatomical structure or other biological matter. In one example, the endoscope may be used in conjunction with a laryngoscope to establish a viewpoint of the larynx of a patient, which may facilitate insertion of an endotracheal tube during an intubation procedure.

In addition to viewing internal structures of the body, the endoscope itself may be used to facilitate variety of medical procedures. For example, an endoscope may include a working channel routed from the endoscope proximal end, through the interior of the endoscope, to the steerable tip, where an opening provides access to an anatomical cavity. The working channel may be used to pass instruments into the anatomical cavity for performing a procedure. As an example, the working channel may be used to perform an endoscopic retrograde cholangiopancreatography (ERCP), in which the endoscope may be navigated into a portion of the GI tract to treat problems associated with the bile and pancreatic ducts. During ERCP, an instrument may be passed through the working channel to open blocked or narrowed ducts, perform a biopsy, or to perform other surgical functions.

One limitation associated with an internally routed working channel is that the endoscope camera system may only provide a single viewpoint of the instrument during the procedure. For instance, the instrument may pass through the endoscope steerable tip and into the anatomical cavity near the camera system, which may provide the camera with a single viewpoint from behind the instrument. In some examples, this single viewpoint may be adequate for completing the procedure, while in other examples, the procedure may benefit from alternative views of the instrument and/or the surgical site. Further, as an instrument is passed within the interior working channel and through the steerable tip, the force applied to advance the instrument may affect the articulation of the steerable tip, thereby affecting the viewpoint of the camera system. Articulation of the steerable tip may also be hindered by a semi-rigid instrument passing through the center of the endoscope.

The technology described herein relates to a collapsible working channel routed nearer to an exterior of the endoscope rather than through the center of the endoscope. When the collapsible working channel is needed, the collapsible working channel may be expanded to provide access to an instrument during an endoscopic procedure, and the collapsible working channel may be collapsed at the conclusion of the procedure. The distal opening of the collapsible working channel is positioned proximally from the endoscope steerable tip, which allows the steerable tip to be articulated independently from the instrument. The endoscope may be configured to articulate the steerable tip to provide additional viewpoints of the instrument, such as a perspective view. The additional viewpoints may improve the accuracy of the procedure and may allow the procedure to be completed more quickly and/or efficiently.

In addition, during an endoscopic procedure, movements of the patient may cause the viewpoint of the endoscope camera system to shift away from the surgical site or other area of interest. For example, the viewpoint may be affected by intentional or unintentional movement of the patient, such as motions associated with respiration, movement of a patient's limb (e.g., an arm, leg, etc.), the patient being rolled or shifted, or other types of movement/motion of the patient. In examples, changes to the viewpoint of the endoscope camera system may result in the clinician pausing the procedure to re-establish the viewpoint or may require the assistance of a second clinician to control the endoscope and maintain the viewpoint.

To help alleviate this issue, the present technology also relates to a virtual anchor feature or mode, which automatically maintains the viewpoint of the endoscope camera system in the presence of movement/motion of the patient's anatomy and/or portions of the endoscope. The clinician may navigate the endoscope into position in an anatomical lumen or cavity and articulate the steerable tip to establish a desired viewpoint. When the virtual anchor feature is enabled, a control algorithm executed by the control device analyzes video image data received from the endoscope camera system. If the control algorithm detects changes in the video image data that indicate a change in the viewpoint of the camera system, the control device automatically generates steering signals that correct for the change and re-establish or maintain the desired viewpoint. In one example, the virtual anchor feature may help maintain the perspective view established during an endoscopic procedure associated with the collapsible working channel. Accordingly, rather than having to use a physical anchor, or in addition to using a physical anchor, the systems of the disclosed technology allow for software controls that maintain a consistent or stabilized view of the desired target or viewpoint.

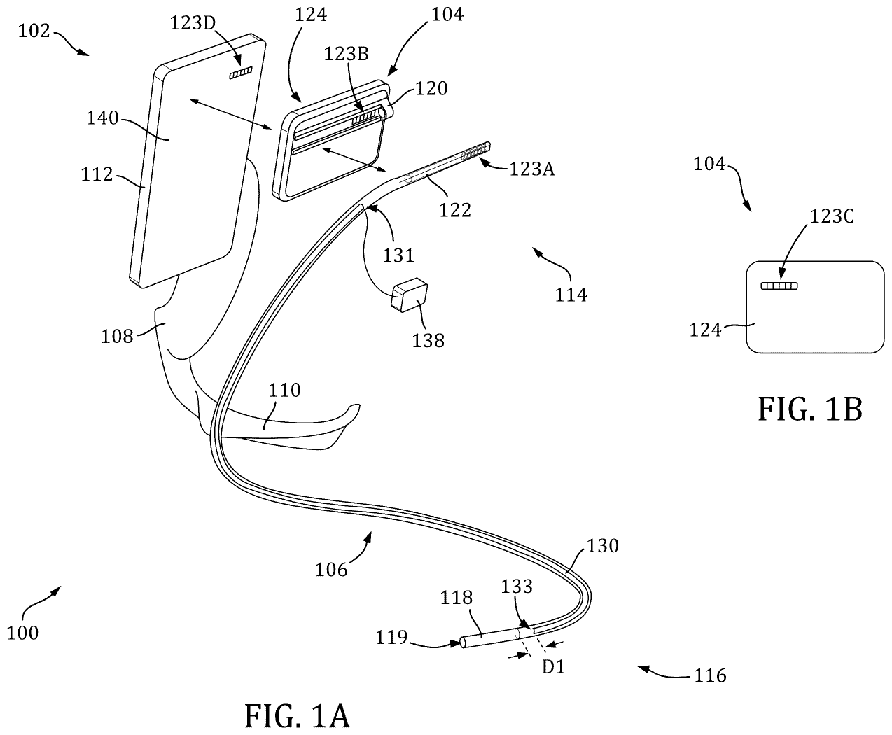

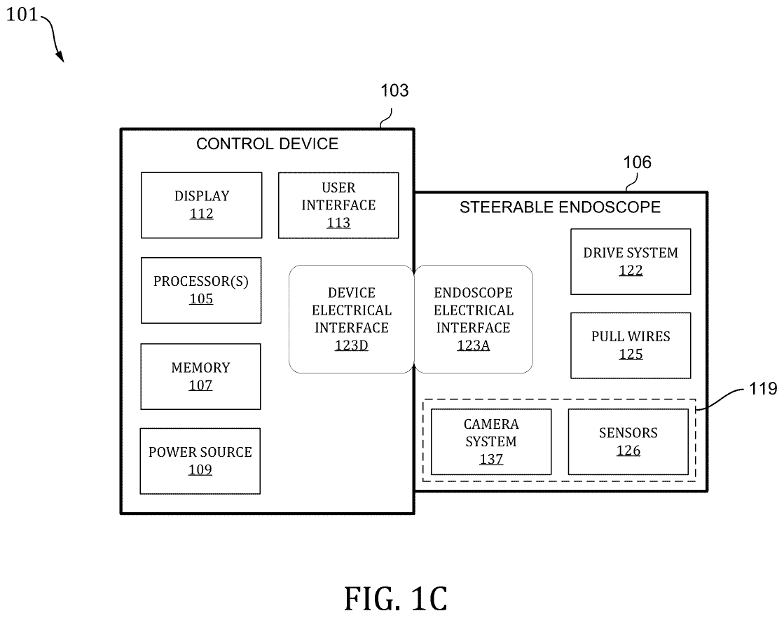

FIGS. 1A-B depict an example medical video system 100 that includes a video laryngoscope (VL) 102 capable of connecting to, and providing steering control of, a steerable endoscope 106, through a detachable cartridge 104. The example video system 100 may be used to perform an intubation procedure or other procedure associated with the upper airway, in which a clinician may prefer to use an endoscope 106 in conjunction with the VL 102 to augment the view of the airway. Example video system 100 is one example where a virtual anchor feature may be used to perform an endoscopic procedure.

The distal end 116 of endoscope 106 includes a steerable tip 118 and accessories 119, which may be used during operation of the endoscope 106. For example, the accessories 119 include a camera system (e.g., a video camera and light source) arranged to capture image data (e.g., video image frames of the airway) from the perspective of the steerable tip 118 during use. The accessories 119 may also include sensors, such as an accelerometer or inertial measurement unit (IMU), which provides measurement data associated with the acceleration, angular velocity, position, and/or other variables associated with the position/orientation/movement of the steerable tip 118.

In one example, the endoscope proximal end 114 may include a drive system 122 that controls the steerable tip 118 via one or more pairs of pull wires (not depicted), which are routed along the interior of the endoscope 106 and connected to different points on the interior of the steerable tip 118 and/or other points of the distal end 116. Each pair of pull wires may be connected to elements of the drive system 122 such that the pull wires of each pair work in opposition to one another to cause articulation of the steerable tip 118 in a movement plane. For instance, the pull wires of a pull wire pair may connect to opposite sides of the steerable tip 118 for causing articulation in a first movement plane (e.g., a left/right movement plane). The endoscope may include a second pair of pull wires arranged to cause articulation of the steerable tip 118 in a second movement plane (e.g., an up/down movement plane). In some examples, the endoscope 106 may include pull wire pairs configured to cause articulation of the steerable tip 118 in additional movement planes, and/or to cause articulation at one or more articulation points (e.g., elbows, joints, etc.) within the steerable tip 118. In other examples, the drive system 122 may cause articulation of the steerable tip 118 by a method other than pull wires.

The endoscope proximal end 114 also includes an electrical interface 123A, through which the endoscope 106 may receive electrical power and may transmit/receive signals to/from the VL 102. For example, the electrical interface 123A provides power and/or steering control signals from the VL 102 to the drive system 122 for controlling the movement of the endoscope steerable tip 118. The electrical interface 123A also provides a source of input power for operating the accessories 119 (such as the camera system, sensors, etc.), and/or other sensors or electronic elements included within the endoscope 106.

Further, the electrical interface 123A provides a data path for transmitting sensor data, video image data, and/or other types of data from the endoscope 106 to the VL 102. For instance, video image data captured by the endoscope camera system may be transmitted to the VL 102 via the electrical interface 123A. The electrical interface 123A may include a plurality of electrical contacts, such as conductive pads, receptacles, pins, balls, ports, and/or other types of electrical contacts that are connected to electrical elements within the endoscope 106 (such as accessories 119) by a plurality of conductors routed within the interior of the endoscope 106. The conductors (not depicted) may include one or more wires, flexible printed circuits (FPCs), and/or other types of electrical conductors suitable for distributing power and establishing signal connection between the electrical interface 123A and electrical elements of the endoscope 106.

To connect the endoscope 106 to the VL 102, the endoscope proximal end 114 is connected to the detachable cartridge 104, which serves as an electrical and/or mechanical interface between the VL 102 and endoscope 106. In one example, the endoscope proximal end 114 slides into a cartridge guide 120, where elements of the endoscope electrical interface 123A make electrical contact and/or mate with corresponding elements of cartridge electrical interface 123B. The guide 120 includes rails or other elements that retain the endoscope 106 to the cartridge 104. In other examples, the endoscope 106 may be connected to, and retained by, the cartridge 104 by another method. As depicted in FIG. 1B, the cartridge rear surface 124 includes an electrical interface 123C for making electrical connection with the VL 102. The electrical interface 123B may be connected to electrical interface 123C within the cartridge 104 by any of a variety of connection methods, such as by electrical wire, rigid or flexible pins, printed circuit board (PCB), FPC, and/or other methods of electrical connection.

The cartridge 104 may be connected and retained to the VL rear surface 140 by any of a variety of methods, such as by permanent magnets located within the VL 102 and/or cartridge 104, or by other elements that apply force between the VL 102 and cartridge 104. When the cartridge 104 is connected to the VL 102, the electrical interface 123C is conductively connected to the VL electrical interface 123D on the VL rear surface 140, which completes the electrical connection between the VL 102, cartridge 104, and endoscope 106.

The VL 102 includes a display 112, a handle 108, and a blade 110. The blade 110 may include or house portions of a VL camera system capable of providing video images from the perspective of the blade 110. The video images may be displayed on the display 112, which may be capable of displaying images from multiple camera systems simultaneously, such as images from the VL camera system and the endoscope camera system. For example, the display 112 may be capable of providing split screen, picture-in-picture, or other method for simultaneously displaying video images. The display 112 may be any of a variety of display technologies, such as liquid crystal display (LCD), light emitting diode (LED), organic light emitting diode (OLED), or other display technology. In examples, the display 112 may be a touch-sensitive display (e.g., a capacitive touch-sensitive display) that allows the steerable tip 118 to be controlled through the display 112. Aspects of the operation of the VL 102 and/or endoscope 106 may also be configured via the display 112, such as video image display preferences and other configurable settings of the example video system 100.

The VL 102 includes additional functions, features, and elements typically associated with a video laryngoscope. For example, the VL 102 includes a power source (e.g., a battery, power regulation circuitry, etc.) for providing power to electrical elements of the VL 102 and endoscope 106. The VL 102 further includes one or more digital processing elements (e.g., a processor) and memory elements (e.g., RAM, ROM, flash memory, etc.) that may be associated with control of the endoscope steerable tip 118 and the processing of data (e.g., video data, sensor data, etc.) received from the endoscope 106. For instance, one or more processors within the VL 102 may translate steering input received from the display 112 (or other element) into control signals for controlling the steerable tip 118, which may further be transmitted to the endoscope drive system 122 via electrical interfaces 123A-D. One or more processors of the VL 102 may also receive video image data from the endoscope 106 and analyze, process, and/or format the video data for viewing on display 112. Additional details are provided below, with respect to FIG. 1C.

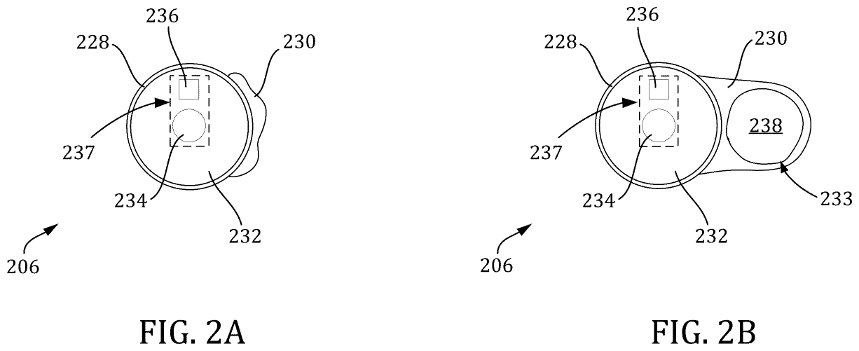

The endoscope 106 further includes a collapsible working channel 130 routed along the exterior length of the endoscope 106. As described below with respect to FIGS. 2A-B, the working channel 130 may remain collapsed while the endoscope 106 is navigated into position in the airway, then expanded as needed during the procedure, such as to insert an instrument or apply a topicalizing agent.

In some examples, the working channel 130 may be expanded by a pneumatic method, such as by manual or automatic inflation, or may be expanded using any of a number of suitable methods. For example, a pump 138 or other type of inflation device may be used to inflate and expand the working channel 130. In other examples, a syringe or other manually controlled device may be used to inject air into the working channel 130 to inflate and expand the working channel 130. Accordingly, the working channel 130 may be selectively expanded and collapsed.

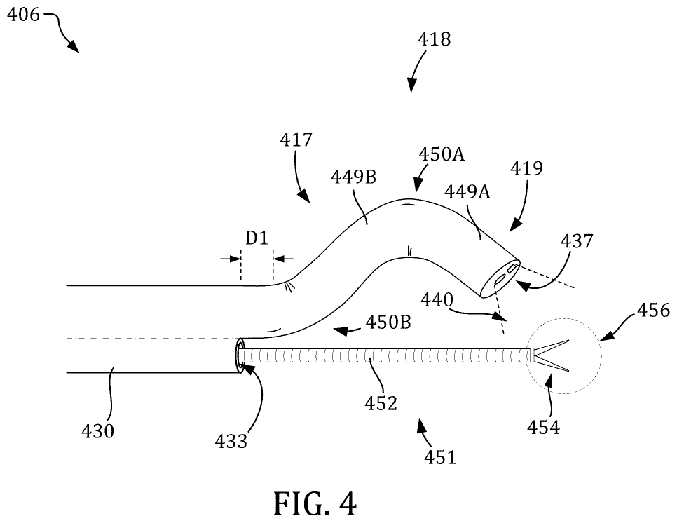

The working channel 130 includes a proximal opening 131 and a distal opening 133. The proximal opening 131 may be suitably spaced from the drive system 122 to allow insertion of an instrument into the working channel 130 when the endoscope 106 is connected to the cartridge 104. The distal opening 133 is offset or spaced proximally from the steerable tip 118 by a distance D1, to avoid interfering with articulation of the steerable tip 118. Additionally, by spacing the distal opening 133 proximally from the steerable tip 118, the steerable tip 118 may be articulated such as to provide a prospective view of an instrument that is extended from the distal opening 133 (depicted in FIG. 4). In some examples, the distance D1 between the distal opening 133 and the steerable tip 118 may be less than 1 cm, while in other examples the distance D1 may be greater than 1 cm, 2 cm, or 3 cm and/or less than 5 cm, 4 cm, or 3 cm.

FIG. 1C is a block diagram of another example video system 101, such as an endoscopic imaging system, in which the steerable endoscope 106 connects directly to a control device 103. While example video system 100 (depicted in FIG. 1A-B) may be used to perform intubations or other procedures associated with the upper airway or anatomy primarily accessed through the mouth, example video system 101 may represent types of control devices 103 and steerable endoscopes 106 used to perform other types of procedures in other portions of a patient's body. For instance, the control device 103 and endoscope 106 may be used to perform procedures associated with the GI tract, such as ERCP or other types of procedures. In some examples, the control device 103 may be similar to, or the same as, VL 102, where functions of the cartridge 104 have been integrated into the VL 102 and the endoscope 106 connects directly to the VL 102.

As described above, the endoscope 106 includes accessories 119, a drive system 122, and pull wires 125. The accessories 119 may include a camera system 137 and sensors 126, among other elements. The camera system 137 includes a light source and camera (further depicted in FIGS. 2A-B) for imaging an anatomical lumen of the body. The sensors 126 may include one or more electronic sensors, such as an IMU, accelerometer, etc.

The pull wires 125 include one or more pull wire pairs that each cause articulation of the steerable tip 118 in a movement plane. In some examples, the pull wires 125 may include a first pull wire pair that causes articulation in a first movement plane (e.g., left/right), and a second pull wire pair that cause articulation in a second movement plane (e.g., up/down). The steerable tip 118 may articulate in the first and second movement planes at a first articulation point, where the steerable tip of the endoscope bends, or articulates, in response to tension applied to the pull wires 125.

Additionally or alternatively, the pull wires 125 may include a pull wire pair connected to cause articulation of the steerable tip 118 at a second articulation point, located proximally from the first articulation point. The combination of the first and second articulation points may allow the steerable tip 118 to articulate in an “S” shape or a serpentine shape (such as depicted in FIG. 4). In one example, the steerable tip 118 may be articulated in a way that orients the camera system 137 to provide a perspective view of a medical instrument inserted into the anatomical lumen through the collapsible working channel 130.

As detailed above, the endoscope drive system 122 may include one or more electric motors (e.g., DC motors) that apply and release tension to the pull wires 125 to cause articulation of the steerable tip 118. In one example, one pair of pull wires 125 may be connected to a rotational element associated with a single electric motor of the drive system 122. Rotation of the motor axle may cause one of the pull wires 125 to shorten (increasing tension), and the opposing pull wire 125 of the pull wire pair to lengthen (decreasing tension), thereby controlling articulation of the steerable tip 118 in a movement plane. The drive system 122 may include drums, gears, and/or other elements suitable for controlling the tension applied to pairs of pull wires 125. In examples, the drive system 122 may include a single electric motor (and associated elements) for each pair of pull wires 125.

In some examples, the drive system 122 may include passive mechanical elements, rather than electric motors or other active electromechanical elements. For instance, the pull wires 125 may be connected to passive elements of the drive system 122 (such as one or more drums) that receive steering forces mechanically transmitted from the endoscope controller (e.g., control device 103). In such examples, the control device 103 may include electric motors that couple to the passive elements of the drive system 122 via a mechanical interface (not depicted) between the control device 103 and endoscope 106.

Electrical elements of the endoscope 106, such as the camera system 137, sensors 126, elements of the drive system 122, and/or other electrical elements, receive electrical power and transmit/receive signals to/from the control device 103 via electrical interfaces 123A-D. As described above, the endoscope 106 may transmit video image and sensor data to the control device 103 via the electrical interfaces 123A-D. In some examples, signals or data (such as clock, enable, timing, and/or other signals) may also be transmitted/received through the electrical interface 123A in order to enable or configure operation of the endoscope 106. The electrical interfaces 123A-D may include active and/or passive circuit elements that support the functioning of the electrical interfaces 123A-D. The electrical interfaces 123A-D include a plurality of electrical contacts, such as those described above, that allow the electrical interfaces 123A-D to be mated together when the endoscope 106 is connected to the control device 103.

The control device 103 includes a power source 109, which may include a battery capable of powering the control device 103 and supplying power to the endoscope 106. The power source 109 may further include analog or digital circuitry associated with control, regulation, and/or distribution of electrical power to elements of the control device 103 and/or endoscope 106. For instance, the power source 109 may include power regulation circuitry.

The control device 103 further includes a display 112 and user interface 113. The display 112 may function substantially as described above, where video image and sensor data received from the endoscope 106 may be displayed on the display 112, and the display 112 may function as touch-sensitive display, capable of receiving steering input.

In some examples, a graphical user interface (GUI) may be provided on the display 112 by the user interface 113 for receiving user input. In examples where the display 112 provides for steering control of the endoscope steerable tip 118 (such as when the display 112 is a touch-sensitive display), the user interface 113 may include a steering feature as part of the GUI. The GUI may include soft menus or similar features that allow a user to configure settings, enable features or functions, set operating parameters, store data (such as user-specific data), and/or modify other user-configurable inputs.

The user interface 113 may also receive input from other types of components, such as buttons, switches, knobs, and/or other input components associated with the control device 103. In some examples, the user interface 113 may receive steering input from an external controller (not depicted) that is operatively coupled to the control device 103 for controlling the steerable tip 118. The user interface 113 may also provide audio alerts, such as through a speaker associated with the control device 103.

Additionally, the control device 103 includes a processor 105, which may include one or more general purpose processors, microprocessors, microcontrollers, graphics processing units (GPUs), digital signal processors (DSPs), and/or other programmable circuits. In examples, the processor 105 may include any combination of commercially available components, and/or custom or semi-custom integrated circuits, such as application specific integrated circuits (ASICs). The processor 105 may include elements needed for control or communication with the display 112, user interface 113, power source 109, and/or device electrical interface 123D. The processor 105 may perform control, interface, communication, or other processing functions by executing instructions that are stored in the memory 107. For instance, the memory 107 may store instructions that, when executed by the processor 105, cause the elements of the example video system 101 to perform operations described herein. In one example, the memory 107 may store portions of one or more algorithms associated with a virtual anchor feature or mode. The memory 107 may include random access memory (RAM), read-only memory (ROM), electrically erasable programmable read-only memory (EEPROM), flash memory or other memory technology.

Further, the processor 105 may be associated with the processing, analysis, and/or display of video image frames received from the camera system 137 through the electrical interfaces 123A-D. For example, the processor 105 may execute a virtual anchor algorithm, where video image frames are analyzed and used to generate steering control signals provided to the drive system 122 for maintaining the viewpoint of the endoscope steerable tip 118, as discussed further below.

The processor 105 may further be associated with translating steering input received from the display 112 or other directional controller into steering signals provided to the drive system 122. For instance, the processor 105 may work in conjunction with elements of the display 112, user interface 113, and/or other elements of the control device 103 to generate steering signals, which are provided to the drive system 122 via electrical interfaces 123A-D.

FIGS. 2A-2B depict a view of the front surface 232 of a distal end of an example endoscope 206, which may be similar to or the same as endoscope 106. The front surface 232 may be plastic, glass, composite, or other type of material, which is sealed against an outer jacket 228. The camera system 237 captures images of the anatomy through the front surface 232.

The camera system 237 includes a light source 236 that illuminates the field-of-view (FOV) of a camera 234. The light source 236 may be a type of LED, lamp, or other type of light-emitting element suitable for use in the example endoscope 206. The camera 234 includes an imaging sensor, such as a charge-coupled device (CCD), complementary metal-oxide-semiconductor (CMOS), or other type of sensor, capable of providing video image frames at a rate and resolution suitable for a clinician to perform endoluminal procedures. For example, the camera 234 may provide video image frames with sufficient resolution for a clinician to discern various features within an anatomical lumen. Further, the camera 234 may provide video image frames at a rate that provides for suitable viewing of movement within the FOV of the camera 234, such as the movement of instruments deployed into the FOV through the collapsible working channel 230.

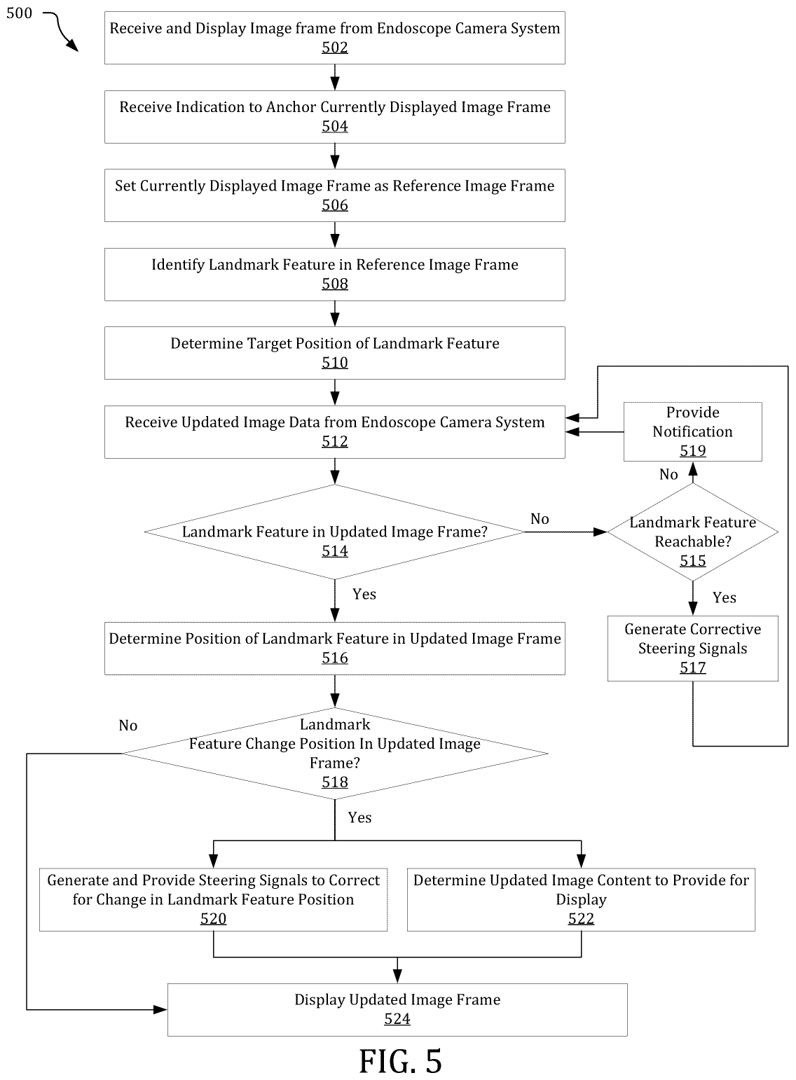

The camera 234, camera system 237, and/or other elements associated with the front surface 232, may also include one or more lenses (not depicted) that modify the appearance of features imaged in the FOV. For example, the camera 234 may include one or more lenses that provide a specific focal length, which may affect which features of the anatomical lumen are in focus in the acquired image frames. In other examples, one or more lenses of the camera 234 may affect the FOV. For instance, the camera 234 may include a wide-angle lens that increases the FOV to capture a larger portion of the anatomical lumen. In one example, the camera 234 may include a fisheye lens which may provide a very wide viewing angle, such as a substantially 180° FOV or greater. In examples where the camera 234 includes a lens that provides an expanded FOV, elements of the external control device (such as the processor 105) may crop the received image frames to display a smaller portion of the FOV than what is acquired by the camera 234. Such a feature is described in more detail below for example method 500 of FIG. 5.

The example endoscope 206 also includes an outer jacket 228, which may be a thin-walled tubular structure that forms a flexible outer sheath around the entirety of, or a substantial portion of, the length of the example endoscope 206. The outer jacket 228 seals the example endoscope 206 while allowing articulation of the steerable tip and flexion along the length of the example endoscope 206. Accordingly, the outer jacket 228 may be fabricated using a material with a high degree of flexibility, such as a thin polyurethane extrusion or other type flexible material.

The working channel 230 may be affixed to the outer jacket 228, such as by adhesive, thermal bonding, or another method of fixation. The working channel 230 may be fabricated using a soft, flexible material, such as any of a variety of elastomers. In some examples, the working channel 230 may be formed as part of the outer jacket 228, such that the working channel 230 and outer jacket 228 are of the same material, and the working channel 230 is continuous with the outer jacket 228. When collapsed, such as depicted in FIG. 2A, the material of the working channel 230 may remain amorphous and compliant, allowing the working channel 230 to bend, flex, conform, etc., as the example endoscope 206 is navigated into position in an anatomical lumen. In the collapsed state, the working channel 230 may present minimal or insignificant changes to the flexibility of the example endoscope 206 and may not significantly affect the ability of a clinician to insert the endoscope 206 into an anatomical lumen.

FIG. 2B depicts the working channel 230 in an expanded state with the channel lumen 238 exposed. When expanded, the working channel 230 may be used to pass instruments from a proximal opening of the working channel 230 (depicted in FIG. 1A), through the channel lumen 238, and through the distal opening 233, where an instrument may access the anatomical lumen or other cavity of the patient.

The working channel 230 may be expanded by a number of suitable methods. In one example, and as briefly described above, the working channel 230 may be pneumatically expandable and include internal cavities that may be filled with air, such as by an external pneumatic device (e.g., pump, syringe), connected to a proximal portion of the working channel 230 or example endoscope 206. For instance, the proximal end of the working channel 230 may include a port for connecting the pneumatic device, or the example endoscope 206 may include a connection port, which is further connected within the example endoscope 206 to inflatable portions of the working channel 230. When appropriate (e.g., at the conclusion of a medical procedure) the working channel 230 may be deflated. In some examples, elastic properties of the material of the working channel 230 may cause the working channel 230 to collapse against the example endoscope 206, returning the working channel 230 to the more compliant state. In other examples, air may be withdrawn from the working channel 230, such as by the external pneumatic device. The withdraw of air may create a vacuum within the working channel 230 that causes collapse of the working channel 230 by atmospheric air pressure. In other examples, the working channel 230 may be expanded/collapsed by other methods, such as by the injection/withdraw of fluid into cavities of the working channel 230, or by other methods for causing expansion/collapse.

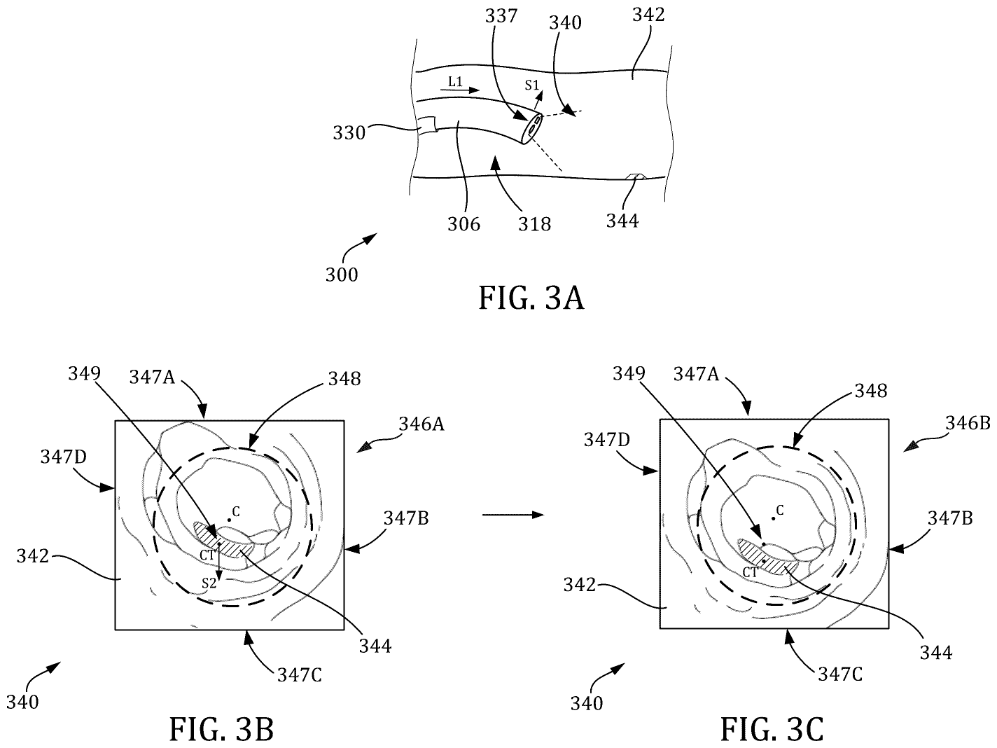

FIG. 3A depicts an example anatomical lumen 300 in which an example endoscope 306 has been navigated. The endoscope 306 may be similar to, or the same as, example endoscopes 106 and 206, described above, and may include the same or similar elements (e.g., steerable tip 318, camera system 337, working channel 330, etc.). The example endoscope 306 includes a camera system 337 that provides video image data of the anatomical lumen 300 to a control device (depicted in FIGS. 1A-C), to which the proximal end of the example endoscope 306 is connected. The video image data is provided to the control device in the form of a sequence of image frames acquired over time, which are displayed on the control device (such as on display 212) in the order received from the example endoscope 306.

FIGS. 3B-C depict image frames 346A-B, respectively, in which anatomical features of the lumen walls 342 are visible within the FOV 340 of the camera system 337. In the sequence of image frames received by the control device, image frame 346A precedes image frame 346B, which is updated with any viewpoint changes that may have occurred in the intervening time between image frames 346A-B. Processing elements of the control device (such as processor 105) may process the received image frames 346A-B for display and/or may store the image frames or portions of the image frames 346A-B in memory for further analysis (such as in memory 107). In one example, image processing elements may crop a portion of the image frames 346A-B and provide image data within the cropped region 348 for display. Image data cropped (e.g., image data that is captured but not displayed) from image frames 346A-B may be stored for additional analysis or discarded. In some examples the cropped region 348 is substantially circular, while in other examples, the cropped region 348 may be another shape, such as substantially rectangular. The cropped region 348 may be concentric about the center C of the image frames 346A-B or may be positioned in another portion of the image frames 346A-B.

The control device may include a virtual anchor feature or mode in which one or more processors of the control device automatically control the steerable tip 318 to maintain the viewpoint of the camera system 337 on a particular region of the anatomical lumen 300. Upon entering the virtual anchor mode, the control device selects one or more of the latest received image frames to serve as a reference image frame or frames. For instance, the control device may use image frame 346A as a reference image frame. The control device may further analyze the reference image frame 346A and automatically identify and select one or more features of the lumen walls 342 as points of reference for maintaining the viewpoint of the camera system 337. For instance, a landmark feature 344 may be automatically identified within the cropped region 348 and used to maintain the viewpoint. The landmark feature 344 may be identified based on attributes of the landmark feature 344 that are identifiable and distinguishable by image processing methods performed by the control device. For example, the landmark feature 344 may include attributes such as distinctive boundaries, color, shading, shape, and/or other attributes that are identifiable/distinguishable by image processing, and that may be used as a reference point for maintaining the viewpoint of the camera system 337. The landmark feature 344 may be identified through feature recognition algorithms or classifiers, such as machine-learning models and/or artificial-intelligence (AI) models (e.g., convolutional neural networks (CNNs), vision transformers). The landmark feature 344 selected by the control device may not be of clinical interest or significance to the clinician or may otherwise not be related to clinical features within the anatomical lumen 300 deemed clinically relevant.

In some examples, the control device may be configured to allow a clinician to identify and manually select the landmark feature 344. For instance, a clinician may select the landmark feature 344 by interaction with the display, such as by tapping on a region of the display that includes the feature or by drawing a shape on the display that encompasses the feature (e.g., with a finger or stylus). The clinician may select a landmark feature 344 that is outside of a region a surgical site, where the clinician may perform a medical procedure with an instrument via the collapsible working channel 330. In examples, the landmark feature 344 may be selected such that a view of the landmark feature 344 is unobstructed by the instrument during the procedure.

In addition, a plurality of features may be identified and selected (either automatically or manually) as landmark features 344. For example, the control device may select primary, secondary, and tertiary (and additional) features that may be used as landmark features 344 by a virtual anchor algorithm to maintain the viewpoint of the camera system 337. The control device may use one or more of the landmark features 344, and/or may switch between landmark features 344 if the view of one or more of the landmark features 344 becomes obstructed during the procedure.

In some examples where the landmark feature 344 is identified automatically by the control device (rather than through manual selection), the landmark feature 344 may lie outside or partially outside of the cropped region 348. In other examples, the control device may select two or more landmark features located inside the cropped region 348, outside the cropped region 348, or a combination of landmark features located inside and outside of the cropped region 348.

The control device may further identify secondary characteristics of the landmark feature 344 in the reference image frame 346A that enable the control device to determine whether positional changes of the landmark feature 344 have occurred between the reference image frame 346A and the updated image frame 346B, such as due to movement(s) of the patient. In one example, the control device may identify the border of the landmark feature 344 and may use the area within the identified border to determine the centroid CT of the landmark feature 344 in the reference image frame 346A. As subsequent image frames (such as updated image frame 346B) are received by the control device, image processing is performed to identify the landmark feature 344 in the updated image frames and determine a centroid CT for each landmark feature 344 in each updated image frame. For instance, the control device may compare the position of the centroid CT in the reference image frame 346A to the position of the centroid CT in updated image frame 346B to determine whether the landmark feature 344 has changed positions within the captured image frames.

While the use of the centroid CT is discussed below as the primary example for clarity, other secondary characteristics representations of the landmark feature 344 may alternatively or additionally be used to determine a positional shift of the landmark feature in the image frames. For example, a functional representation of the boundary of landmark feature 344 may be generated and tracked between frames. In other examples, a bounding box for the landmark feature 344 may be generated, and the position of the bounding box may be tracked across image frames.

In some examples, the control device may determine whether the position of the landmark feature 344 has changed based on the position of the centroid CT relative to features of the image frames 346A-B. For instance, the control device may analyze the reference image frame 346A to determine the distance and/or angular direction between the centroid CT and the center C of the reference image frame 346A. The distance may be determined using the number of pixels (dot-pitch) between features or may be determined using another measure of distance (e.g., metric distance). Additionally or alternatively, the control device may analyze the reference image frame 346A to determine the distance between the centroid CT and one or more of the image frame edges 347A-D. In other examples, the control device may use other features of the reference image frame 346A, such as the distance between the centroid CT and the corners of the reference image frame 346A, or may analyze the reference image frame 346A for other distance metrics for determining the position of the centroid CT. The control device may use the determined distances within the reference image frame 346A to establish a target position 349 of the centroid CT, to which the position of the centroid CT in subsequent image frames may be compared. When subsequent image frames are received (such as updated image frame 346B), the control device establishes the position of the centroid CT of the landmark feature 344 by determining the distances between the centroid CT and the features of the image frame described above (e.g., distance to the center C, edges 347A-D, etc.). The control device then compares the position of the centroid CT in the subsequent image frames to the target position 349 in the reference image frame 346A, to determine whether the viewpoint of the landmark feature 344 has changed.

As an example, the steerable tip 318 of the endoscope 306 may be deflected in a direction S1 (depicted in FIG. 3A) due to movement of the patient and/or the example endoscope 306. The deflection may cause the landmark feature 344 to deflect in a direction S2 (depicted in FIG. 3B) relative to the target position 349 within to the reference image frame 346A. The control device may detect the deflection by comparing the position of the centroid CT in the updated image frame 346B to the target position 349 of the centroid CT in the reference image frame 346A. For example, the control device may determine that in the updated image frame 346B, the vertical distance between the centroid CT and the bottom edge 347C has decreased (relative to the target position 349), and that the vertical distances between the centroid CT and center C, and between the centroid CT and top edge 347A have both increased. In response, the control device may provide steering signals to the example endoscope 306 to correct for the deflection by articulating the steerable tip 318 in a direction and magnitude to correct for, or compensate for, the deflection. The control device may analyze additional updated image frames to determine whether articulation of the steerable tip 318 has restored the centroid CT to the target position 349.

The control device may use multiple measures of distance to determine whether the centroid CT has deflected from the target position 349. As described above, the control device may use vertical distance(s) from the centroid CT to the center C and/or to the top or bottom edges 347A, C, or may use horizontal distance(s) from the centroid CT to the center C and/or to the left or right edges 347B, D. In some examples, the control device may use a combination of vertical and horizontal distances to determine changes in the position of the centroid CT relative to the target position 349. In other examples, rather than horizontal/vertical (or x-y) distance measures, the control device may use a polar coordinate system, or other coordinate system to determine changes in the position of the centroid CT. In still other examples, the control device may compare the absolute position of the centroid CT to the target position 349, rather than using the relative measures described above.

Additionally or alternatively, the control device may compare the position of the centroid CT to the edge or center of the cropped region 348 to determine whether the position of the centroid CT has changed relative to the target position 349. For example, the position of the centroid CT may be compared to reference points on the edge of the cropped region 348 to determine relative changes from the target position 349. In other examples, the centroid CT may be compared to other aspects or features of the cropped region 348 to determine positional changes.

While articulating the steerable tip 318 to correct for the deflection S1/S2, the control device may articulate the steerable tip 318 until the centroid CT is within a distance tolerance of the target position 349 in the reference image frame 346A. For example, following a deflection of the steerable tip 318 the control device may articulate the steerable tip 318 until the distance between the centroid CT and center C is within 10% of the distance between the target position 349 and the center C in the reference image frame 346A. In some examples, the control device may use a tolerance of 5%, or may use a lower or higher tolerance value. In other examples, the control device may use a measure of absolute tolerance (e.g., absolute distance or number of pixels) to determine whether the centroid CT is repositioned at the target position 349. For example, the control device may articulate the steerable tip 318 until the centroid CT is within 0.1 mm of the target position 349 (where a metric distance measure is used) or is within 5 pixels of the target position 349 (where dot-pitch distance is used). In other examples, a larger or smaller tolerance may be used to determine whether the viewpoint has been restored.

Similarly, the control device may not correct for small deflections of the steerable tip 318, where the position of the centroid CT is changed by less than a distance tolerance from the target position 349. For instance, the control device may not correct for deflections that result in less than a 10% or 5% change in the position of the centroid CT relative to the target position 349. In examples, the control device may not correct for small deflection less than a specified distance, such as 0.1 mm, 5 pixels, or less than another measure of distance.

Tolerances may be specified through user settings provided by the control device. The tolerances specify the amount of image movement deemed acceptable to the user/clinician. Higher tolerances result in larger allowable shift in the viewpoint of the steerable tip 318 and lower tolerances permit smaller shifts in the viewpoint, which may provide a steadier view of the anatomical lumen 300.

The control device may use other aspects or features of the landmark feature 344 to determine whether the position of the landmark feature 344 in the updated image frame 346B has changed relative to the reference image frame 346A. For instance, in examples where the landmark feature 344 is elongate, the control device may determine major and minor axes of the landmark feature 344 and use the axes to determine whether the orientation of the landmark feature 344 has changed between image frames 346A-B. In one example, the major and minor axes may be used to determine whether the steerable tip 318 has undergone a rotational deflection, such as by determining whether the direction of the axes have rotated relative to the reference image frame 346A. In examples, where the steerable tip 318 is configured to correct for rotational deflections, the steerable tip 318 may be articulated accordingly. In other examples, the device may use still other aspects or features of the landmark feature 344, such as surface area, shading (or level of illumination), and/or other aspects or features of the landmark feature 344 to determine whether the viewpoint of the steerable tip 318 has changed relative to the viewpoint provided in the reference image frame 346A.

Alternatively or additionally, the control device may correct for deflections of the steerable tip 318 by shifting the portion of the updated image frame 346B displayed by the control device, rather than by articulating the steerable tip 318. For instance, the control device may acquire image data from the full image frames 346A-B, but as described above, may only display the portions of the image frames 346A-B that appear in the (smaller) cropped region 348. As such, the cropped region 348 may be adjusted to cause the landmark feature 344 to appear stable or anchored to the clinician viewing the display screen. Accordingly, small deflections may be handled by shifting the cropped region rather than having to steer the tip 318. In some examples, a deflection of the steerable tip 318, or movement of the patient, causes the landmark feature 344 to be shifted out of the initial cropped region 348, but still within the larger updated image frame 346B, which would previously cause the landmark feature 344 to no longer be displayed. With the technology discussed herein, the landmark feature 344 may still be detected in the updated image frame 346B (outside of the initial cropped region), and the cropped region may be shifted for the updated image frame 346B to bring the landmark feature 344 back into view and in substantially the same position on the display as shown for the prior image frame. Additional details are provided below in example method 500, depicted in FIG. 5.

In some examples, the camera system 337 may include lens or other elements that allow the camera system 337 to image a larger FOV 340, which may increase the image content acquired in the image frames 346A-B and/or may increase the size of the image frames 346A-B relative to the cropped region 348. The additional image content may allow the control device to correct for larger deflections of the steerable tip 318 without the need for physical steering of the tip 318. By correcting for the deflection of the steerable tip 318 using image processing methods, rather than articulation of the steerable tip 318, the control device may reduce or eliminate a portion of the latencies associated with correcting/maintaining the viewpoint of the camera system 337, resulting in a faster response to deflections. A benefit of decreasing the response latency is that the control device may provide image content from the cropped region 348 that appears more steady or stable when displayed.

Additionally or alternatively, the control device may use the entirety of the image frames 346A-B to determine changes in the viewpoint of the steerable tip 318, rather than using only a portion of the image frames 346A-B, such as the landmark feature 344. For instance, the control device may perform a full comparison of all (or most of) the pixels acquired in the reference image frame 346A and updated image frame 346B. Such a comparison may include comparing a substantial portion of the characteristics or attributes of the features (e.g., shaded regions, boundaries between features, etc.) present in the image frames 346A-B. The control device may correct for deflections by providing steering signals to articulate the steerable tip 318 accordingly, or may shift the image content to display a corrected image, as described herein. The processing of the full image frames 346A-B may include the use of additional computational resources within the control device, such as one or more additional processing elements and/or the use of a greater proportion of the processing elements. The additional processing may also result in increased power consumption by the control device, increased response latency to deflections of the steerable tip 318 (due to increased computational burden), and/or other performance effects. However, processing of the full pixel comparison may eliminate a requirement for identification of a landmark feature 344 within the image frames 346A-B, which may conserve computational resources in a different manner.

In examples, the control device may also, or alternatively, use sensor data to detect deflections of the steerable tip 318. The sensor data may be provided by one or more sensors located in the steerable tip (e.g., sensors 126), such as by the IMU at the distal end of tip 318. When the virtual anchor feature is enabled, data provided by the IMU may indicate a change in the orientation of the steerable tip 318 from an initial set point or anchor point set when the virtual anchor is enabled. For instance, rotation or deflection of the steerable tip 318 may cause the camera to point away from the desired viewpoint. The control device may use data received from the IMU to provide corrective steering signals to the endoscope 306 to return the steerable tip 318 back to its anchor position. In some examples, data from the IMU may be used to augment the image processing methods described above to correct for deflections of the steerable tip 318. For example, large and/or rapid deflections of the steerable tip 318 may result in the landmark feature 344 shifting completely out of the updated image frame 346B. In such an example, the control device may be unable to correct for the deflection using image processing alone, since the landmark feature 344 is not available as a reference for providing corrective steering signals. Data from the IMU indicates the magnitude and/or direction in which the steerable tip 318 was deflected, which may indicate the direction in which the landmark feature 344 may have left the image frame (e.g., the bottom edge 347C, the left edge 347D, etc.).

In addition, IMU data may indicate instances where the deflection of the steerable tip 318 is no longer correctable by the methods described herein. For instance, the example endoscope 306 may have moved into or out of the patient along the proximal-distal (or longitudinal) axis of the anatomical lumen 300, such as in direction LI depicted in FIG. 3A. The movement may be large enough that the steerable tip 318 is moved substantially past the landmark feature 344, to the extent that no amount of articulation of the steerable tip 318 may cause the viewpoint provided in the reference image frame 346A to be restored. For instance, if the endoscope has been advanced distally past the landmark feature 344, no amount of articulation may allow for the camera to be again pointed at the landmark feature 344. Data from the IMU may indicate that a large longitudinal displacement has occurred, and the control device may provide an indication to the clinician.

FIG. 4 depicts an example endoscope 406 articulated to direct the FOV 440 of the endoscope camera system 437 toward an anatomical region of interest 456. The example endoscope 406 may be similar to, or the same as, example endoscopes 106, 206, etc., described above. The example endoscope 406 includes a steerable tip 418, which includes a bendable region 417 and an accessory region 419. The accessory region 417 may include elements associated with the camera system 437, sensors (e.g., an IMU), and/or other elements, and may be substantially more rigid than the bendable region 417 or may otherwise be designed to exhibit minimal flexion relative to the bendable region 417. The bendable region 417 may include a first articulation point 450A and a second articulation point 450B, such that the steerable tip 418 may be articulated in an “S” shape or serpentine shape. Accordingly, the bendable region has two articulating segments, including a first articulating segment 449A between the first articulation point 450A and the distal end and a second articulating segment 449B adjacent to the first articulating segment 449A and between the first articulation point 450A and the second articulation point 450B.

The example endoscope 406 further includes a collapsible working channel 430 affixed to the exterior of the example endoscope 406, such as to an outer jacket of the example endoscope 406 (e.g., outer jacket 228). In examples, the working channel 430 may be integrated or formed as part of the example endoscope 406. The working channel 430 is depicted in FIG. 4 in the expanded state, where the interior lumen (e.g., lumen 238) receives a medical instrument 451. The working channel 430 may be expanded by a variety of methods, such as by a pneumatic method or other method described above.

When the working channel 430 is expanded, the instrument 451 is inserted into a proximal opening of the working channel 430 (e.g., proximal opening 131), which remains outside the body of the patient. The instrument 451 is passed through the working channel 430 and through the distal opening 433 into an anatomical lumen or other cavity of the patient. The distal opening 433 is located proximally from the steerable tip 418, a distance D1 from the bendable region 417, which allows the steerable tip 418 to be articulated without affecting the instrument 451, and without being substantially affected by the instrument 451. Further, by locating the distal opening 433 outside of the bendable region 417, the steerable tip 418 may be articulated to provide a perspective view of the instrument 451 as described herein.

The instrument 451 may include a surgical tool 454 attached at the distal end of flexible tool extension 452, which allows the instrument 451 to follow bends and curves in the working channel 430 associated with the placement of the endoscope 406 within the patient's body. The instrument 451 is extended from the distal opening 433 to position the surgical tool 454 at the region of interest 456, where the surgical tool 454 may be used to perform a medical procedure. For example, the surgical tool 454 may be used to remove tissue or other biological matter, extract a tissue sample, repair a portion of the anatomy, or perform some other medical/surgical function. The steerable tip 418 is articulated to provide a perspective view of the surgical tool 454 and region of interest 456 while the procedure is performed, which may improve the ability of the clinician to perform the procedure accurately and efficiently.

The endoscope 406 is connected to a control device (e.g., control device 103) that may include a virtual anchor feature that automatically maintains the view of the camera system 437 on the region of interest 456. The control device may use an anatomical landmark feature visible within the FOV 440 (e.g., landmark feature 344) to maintain the view of the region of interest 456, as described above. For example, deflections of the steerable tip 418 that change the view of the region of interest 456 may be detected by a virtual anchor algorithm executed on the control device, which may correct for the deflection and restore or maintain the view of the region of interest 456 by sending appropriate steering signals to the example endoscope 406.

The virtual anchor feature may maintain the view of the region of interest 456 even when the surgical tool 454 is present in the FOV 440 and captured in the acquired image frames. In some examples, the surgical tool 454 may be used to perform a procedure in the region of interest 456 where the surgical tool 454 does not obstruct the view of a landmark feature within the FOV 440 of the camera system 437. In such examples, the virtual anchor feature may function irrespective of the presence or absence of the surgical tool 454.

In examples where the surgical tool 454 may obstruct or partially obstruct the view of a landmark feature, the control device may identify an alternative landmark feature in the reference image frame to use as a point of comparison for detecting deflections of the steerable tip 418. In some examples, the control device may select an alternative landmark feature from a set of landmark features that are identified during processing of the reference image frame at the time the virtual anchor is enabled. For instance, the control device may identify a primary, a secondary, and/or a tertiary landmark feature in the reference image frame when the virtual anchor feature is enabled. The control device may use the primary landmark feature when the surgical tool 454 is absent from the FOV 440. The control device may automatically switch to the secondary or tertiary landmark features (or may use both) when the surgical tool 454 is present in the FOV 440. The control device may be capable of automatically identifying the surgical tool 454 within acquired image frames and switching to alternative landmark features as needed.

The virtual anchor feature or mode may allow the clinician to perform the medical procedure using the instrument 451 while substantially disengaging from the control device. For instance, the virtual anchor feature may allow the clinician to set down the control device and use both hands to operate the instrument 451, while observing the action of the surgical tool 454 on a display of the control device. In some examples, the virtual anchor feature may allow a single clinician to perform a procedure using both the example endoscope 406 and instrument 451.

FIG. 5 depicts an example method 500 for restoring and maintaining the viewpoint of an endoscope camera system in response to a deflection of the endoscope steerable tip. The example method 500 may be performed by elements of a control device (e.g., control device 103), such as by one or more processors (e.g., processor 105) or associated system, or by other suitable elements, modules, systems, or combinations thereof. For instance, the memory may store instructions that, when executed by the one or more processors, cause control device to perform one or more of the operations in method 500. The example method may rely on the use of memory or storage elements (e.g., memory 107) during processing or analysis, or for data retention or storage.

At operation 502, the control device receives image data from the endoscope camera system (e.g., camera system 137, 237, etc.), such as through an electrical interface (e.g., electrical interface 123A-D) that electrically connects the endoscope and control device. The image data may be received in the form of a sequence of image frames, each of which includes image content acquired by the camera system from within an anatomical lumen or cavity of a patient's body.

The control device processes the received image frames for display and/or stores the image frames for further analysis. The control device may crop portions of the received image frames to a particular viewing format or display size. For instance, the image frames may be received by the control device in a substantially rectangular format (e.g., image frames 346A-B), and the control device may crop each image frame to form a substantially circular region (e.g., cropped region 348) or a smaller rectangular region. The control device may store the received (un-cropped) image frames, the cropped image frames, and/or the cropped portions of the received image frames. The control device provides the image frames or cropped image frames to a display of the control device (e.g., display 112) for visualization.

At operation 504, the control device receives an indication to anchor the currently displayed image, thereby enabling or activating the virtual anchor feature or mode. The indication may be received through as an input portions of a user interface associated with the control device (e.g., user interface 113). In some examples, the input may be provided through the display, such as in examples where the control device provides a touch-sensitive display. In other examples, the input may be received through user interaction with one or more buttons, switches, knobs, or other types of elements suitable for receiving the input. In examples where the control device includes a defined handle (e.g., handle 108), the handle may include a transducer which generates an electrical signal when a user's grip is fully or partially released from the handle. The received signal from the handle may be used to enable the virtual anchor feature.

At operation 506, upon receiving input to enable the virtual anchor feature or mode, the currently displayed image frame is set as a reference image frame (e.g., reference image frame 346A), which is stored for comparison against subsequently received updated image frames. The image content within the reference image frame represents the viewpoint to be maintained. Accordingly, the reference image frame may also be referred as the anchor frame.

At operation 508, a landmark feature is identified in the reference image frame. The landmark feature may be used for comparing the image content of the reference image frame to the image content of subsequently received updated image frames to detect changes in the viewpoint of the camera system. The landmark feature may be automatically identified by the control device based on attributes of the landmark feature that are identifiable and distinguishable by image processing methods. For example, the landmark feature may include attributes such as distinctive boundaries, color, shading, shape, and/or other attributes that may help identify the landmark feature and/or distinguish the landmark feature from other anatomical features included in the image frame. In some examples, two or more landmark features may be identified and one or more of the identified landmark features may be used as a reference point for comparing the viewpoint between the image frames.

The landmark feature may be identified by the control device using a feature identification model, which may analyze the received image frame for the above attributes and identify a region or area to serve as a landmark feature. In examples, the model may include the use of Haar cascade classifiers, Support Vector Machines (SVMs), convolutional neural networks (CNNs), or other types of classification and/or detection methods or algorithms suitable for identifying one or more landmark features within the image content of the reference image frame. Some examples for identifying objects within endoscopic images are provided in U.S. patent application Ser. No. 18/050,013, titled “Endoscope with Automatic Steering,” and filed on Oct. 26, 2022, which is incorporated herein by reference in its entirety. In examples, the identified landmark feature(s) may include areas or features of the anatomical lumen or cavity deemed clinically relevant or interesting to a clinician.

Identifying one or more landmark features may further include identifying secondary characteristics of each landmark feature that enable the control device to detect changes in the landmark feature in subsequently received image frames, such as changes in position or orientation of the landmark feature in the image frame relative to the reference image frame. In some examples, the centroid, surface area, or other secondary characteristics of the landmark feature may be identified. The control device may determine the centroid, for example, by first establishing the border of the landmark feature, then using the area within the border of the landmark feature to determine the geometric center of the landmark area. In examples where the landmark feature is elongate, the major and minor axes of the landmark feature may be determined, in addition to other secondary characteristics of the landmark feature.

In some examples, one or more landmark features are be identified by the clinician via the control device. For instance, a clinician may select the landmark feature by interaction with the display, such as by tapping on a region of the display that includes the feature, drawing a shape on the display that encompasses the feature, or by another method for highlighting the desired landmark feature (e.g., with a finger or stylus). After one or more landmark features have been identified by the clinician via input into the control device, the control device may automatically identify and/or determine secondary characteristics of the landmark feature, such as the centroid or other secondary features.

At operation 510, the control device determines the position and/or orientation of the landmark feature within the reference image frame, such as by using the secondary characteristics identified or determined at operation 508. For instance, where a centroid of the landmark feature has been identified, the position of the centroid in the reference frame may be determined and stored. The position of the centroid in the reference image frame may be referred to as the target position. In some examples, the absolute position of the centroid within the reference image frame may be used as a target position, such as an absolute horizontal and vertical pixel location within the reference image frame. In other examples, the relative position of the centroid may be used as the target position, such as the horizontal and vertical position of the centroid relative to the center of the reference image frame or relative to one or more edges or corners of the reference image frame. In still other examples, the target position of the centroid may be determined relative to other features of the reference image frame or image content of the reference image frame. The relative position of the centroid may be determined in terms of dot-pitch (in examples where distance is referenced in terms of pixels), metric distance, or other measures of distance. In examples where image frames are cropped for display, the position of the centroid within the cropped image frame (absolute or relative position) may be used as the target position.

Similarly, in examples where the surface area of the landmark feature, major/minor axes of the landmark feature, and/or other secondary characteristics of the landmark feature are used to detect changes in the landmark feature in subsequently received image frames, other measures may be used to establish a target position or target orientation of the identified secondary features of the landmark feature.

At operation 512, the control device receives an updated image frame from the endoscope camera system at a subsequent point in time. At operation 514, the control device attempts to identify the same one or more landmark features in the updated image frame. The one or more landmark features that are attempted to be identified at operation 514 are the same one or more anatomical landmark features that were identified in operation 508.

If the landmark feature(s) cannot be identified in the updated image frame at operation 514, the method flows to operation 515. At operation 515, a determination may be made as to whether the landmark feature is reachable through articulation of the steerable tip. For example, the movement of the distal tip of the endoscope between the time of the capture of the reference frame and the time of the capture of the updated image frame may be determined based on the sensor data from the sensor(s) at the distal tip (e.g., the IMU). A determination may be made based on the type, direction, and or magnitude of the detected movement whether the tip is capable of articulation to bring the landmark feature back into the FOV of the camera.

If the landmark feature can be brought back into view through articulation, the method 500 flows to operation 517 where corrective steering signals are generated that cause the distal tip to be steered such that the landmark feature is brought back into the FOV. The steering signals may be generated based on the sensor signal(s) that indicate the movement of the distal tip during between the time of captures of the reference frame and the updated image frame. Once the distal tip has been steered according to the corrective steering signals, the method 500 flows back to operation 512 where additional updated image frames are captured and analyzed.

If, at operation 515, the control device determines that the landmark feature is not reachable through articulation of the tip, the method flows to operation 519 where a notification may be generated. In one example, movement of the patient and/or endoscope may result in a deflection of the endoscope steerable tip that may not be correctable by further articulation. For instance, the endoscope may be advanced distally (longitudinally) into an anatomical lumen or cavity of the patient to the extent that further articulation of the steerable tip may be unable to restore the viewpoint of the reference image frame. In such an example, the control device may be configured to provide a notification to the clinician. The notification may be provided in the form of an audio and/or visual alert. For example, the control device may include a speaker that broadcasts an audible alert. Additionally or alternatively, the control device may provide a visual alert, such as through the display of the control device, or through an LED, lamp, or other type of visual indicator. The control device may further display a written message associated with the notification. For instance, the written message may indicate the reason for the loss of viewpoint, and/or may suggest corrective action to restore the viewpoint, such as withdrawing the endoscope in the proximal direction to restore the viewpoint.

Returning to operation 514, in examples where the one or more landmark features are identified in the updated image frame, the control device may also identify or determines secondary characteristics of the landmark feature(s), such as the centroid or other secondary characteristic. The control device may further identify the position of the centroid (or other characteristic of the landmark feature) within each updated image frame for which the centroid was determined. The control device may identify the position, orientation, or other metric for any secondary characteristic of the landmark feature suitable for comparing the updated image frames to the reference image frame. In examples where the landmark feature(s) is identified, the method then flows to operation 516.

At operation 516, the control device determines the position of the landmark feature(s) in the updated image frame. As described above, the absolute or relative position of the centroid (or other secondary features of the landmark feature) may be determined and used to represent the position of the landmark feature in the updated image frame. Additionally or alternatively, the orientation of secondary characteristics of the landmark feature(s) may be determined and used to represent the position of the landmark feature in the updated image frame.

At operation 518, the control device determines whether the landmark feature changed positions in the updated image frame, relative to the reference image frame. For instance, a determination is made as to whether there is a difference between the first position of the landmark feature in the reference frame and the second position of the landmark feature in the updated frame. In one example, the determination may be made by comparing the position of the centroid in the updated image frame to the target position in the reference image frame. For instance, the control device may compare the horizontal and/or vertical distances (e.g., dot-pitch, metric distance, etc.) between the centroid of the landmark feature in the updated image frame and the target position to determine whether the position of the landmark feature has changed. In another example, the control device may compare the position of the landmark feature using relative positions, such as by comparing the position of the landmark feature in the updated image frame relative to the center of the updated image frame, to the position of the target position relative to the center of the reference image frame. In another example, the control device may compare the positions of the landmark feature in the updated and reference image frames (using the position of the centroids) relative to the edges, corners, or other portions of the image frames.

In still other examples, the control device may use the orientation of the landmark features in the updated and reference image frames to determine whether the landmark feature changed positions in the updated image frame. For instance, in examples where the landmark feature is elongate, and major and minor axes have been determined for the landmark feature, the control device may determine whether the major and minor axes have undergone rotation relative to the major and minor axes of the landmark feature in the reference image frame. In other examples, the control device may use a combination of landmark feature position, orientation, or some other metric to determine whether the landmark feature changed positions between updated image frame and reference image frame.

In addition, the control device may apply a distance threshold in determining changes to the position of the landmark feature. For example, the control device may determine that the landmark feature in the updated image frame is displaced from the target position by less than a specified distance threshold, such as by less than a specified number of pixels or by a metric unit of length. In one example, the distance threshold may be less than 5 mm, 4 mm, 3 mm, or 2 mm. In examples where the positional changes are compared on a relative basis, such as by comparing the landmark position relative to the center of the image frame, the control device may use a tolerance specified as a percentage. For instance, the control device may use a threshold of less than 10%, 5%, or smaller to determine positional changes. When the landmark feature is within the tolerance, the control device may determine that landmark feature did not substantially change positions, and when the landmark feature is outside the tolerance, the control device may determine that landmark feature did change positions. The control device may provide a method by which the clinician may specify the tolerance, such as through a user interface of the control device.

In examples where the control device determines that the landmark feature in the updated image frame did not substantially change from the target position, the method 500 proceeds “no” to operation 524, which is described further below. In examples where the control device determines that the landmark feature in the updated image frame did change from the target position, method 500 proceeds “yes” to operation 520 or operation 522.

Operations 520 and 522 provide virtual anchoring techniques that may be provided through steering correction of the endoscope (operation 520) or through image correction (operation 522). In some examples, both operations 520 and 522 may be performed. In other examples, only one of operations 520 and 522 may be performed.

At operation 520, the control device corrects for changes in the position of the landmark feature by causing articulation of the steerable tip. In such examples, at operation 520, the control devices uses the position of the landmark feature in the updated image frame to automatically generate steering signals, and then provide those steering signals to the endoscope, which articulates the steerable tip accordingly. In some examples, the control device may use the steering signals to directly steer the tip of the endoscope through motors or actuators within the control device. The steering signals are based on the distance between the positions of the landmark feature in the updated image frame and the reference image frame. For example, larger changes in the position of the landmark feature may correspond to steering signals that cause larger adjustments to the articulation of the steerable tip.

Alternatively or additionally to correcting for a change in the position of the landmark feature by causing articulation of the steerable tip, the control device may adjust the image captured by the endoscope in operation 522. For instance, at operation 522, the control device may determine which content in the updated image frame should be displayed within the cropped image frame in order for the landmark feature to appear at the target position when displayed. For example, following a deflection of the endoscope and/or the endoscope steerable tip, the landmark feature may be shifted away from the target position, as determined above. Image content that was previously positioned within the cropped image frame (which may include the landmark feature) may be shifted to the region outside the cropped image frame, and other image content that was previously in the region outside the cropped image frame may be shifted into the cropped image frame. Such an example is depicted in FIGS. 3B-C and discussed above. The control device may receive the full image data acquired by the camera system within the updated image frame, including image data appearing in the region outside of the cropped image frame. Based on the target position and the position of the landmark feature in the updated image frame, the control device determines the image content from the updated image frame that should appear in the cropped image frame, so that the landmark feature appears at the target position when displayed. Thus, the control device may use image content that would ordinarily be cropped from display to correct for the deflection by shifting the cropped region relative to the full image frame of the updated image frame.

In one example, the control device may determine the image content that should appear in the cropped image frame using the centroid identified at operation 514. For instance, the control device may use the dimensions of the cropped image frame (e.g., the radius/boundary of cropped region 348) and the position of the centroid relative to the center of the cropped image frame to determine the image content around the centroid that should be displayed, so that the content of the cropped image frame matches the content of the reference image frame. In other examples, the control device may use other secondary characteristics of the landmark feature to determine which content in the updated image frame should be provided for display.

In some examples, the camera system may include a type of wide-angle lens that increases the portion of the anatomical lumen or cavity appearing in the FOV of the camera system, which may increase the image content in the region outside of the cropped image frame. By increasing the image content in the region outside the cropped image frame, the control device may be capable of correcting for larger deflections of the endoscope and/or endoscope steerable tip, since more image data may be available for display.

In further examples, the control device may determine the image content that should be displayed in the cropped image frame when a surgical instrument is present in the updated image frame. As described above, when the surgical instrument obscures portions of the landmark feature (e.g., the centroid), the control device may use other secondary characteristics, other landmark features, or methods for identifying image content that should be displayed in the cropped image frame.

At operation 524, the control device displays the updated image frame. In examples where the landmark feature is substantially positioned at or near the target position in the updated image frame (proceeding from operation 518), the updated image frame displayed by the control device substantially matches the reference image frame. In examples where the control device generates and provides corrective steering signals to the endoscope (proceeding from operation 520), the updated image frame displayed by the control device does not match the reference image frame. Corrections to the viewpoint of the camera system may be acquired in subsequently received image frames, following articulation of the steerable tip in accordance with the corrective steering signals. In examples where the control device corrects the cropped image frame shifting the cropped region (operation 522), the updated image frame displayed by the control device may substantially match the reference image frame.

The control device may proceed back to operation 512, where the control device receives a subsequent image frame with updated image content acquired since the previous image frame and may generate steering signals to further correct the viewpoint. The process of providing corrective steering signals to the endoscope at operation 520 and verifying the effect in a subsequently received image frame at operations 512-518 may form a feedback control loop between the control device and the endoscope. Thus, at operation 520, the control device may generate steering signals in accordance with a control algorithm, which may be part of the virtual anchor feature or mode.

In some examples, the control device may use a subset of the updated image frames received, rather than all of the updated image frames received, to determine changes in the position of the landmark feature and provide corrective steering signals. For example, the control device may receive and store (or discard) an updated image frame but may not identify landmark features and secondary characteristics, may not determine whether the image content of the update image frame indicates a change in the viewpoint of the camera system, may not display the received image frame, and/or may not perform any further analysis or processing with the updated image frame. In examples, the control device may process one out of every two updated image frames received, or in some examples may process one out of every three, four, or more updated image frames received. Processing fewer updated image frames may slow the response of the control device to deflections of the steerable tip but may reduce power consumption by the control device.

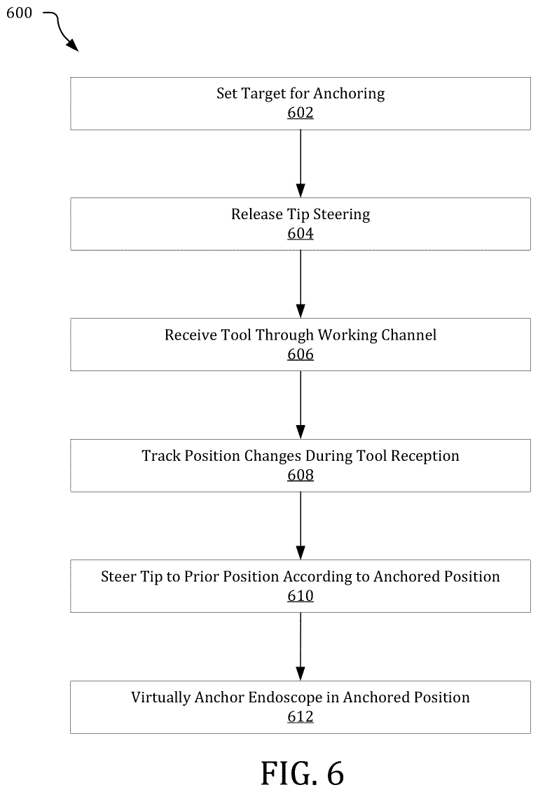

FIG. 6 depicts another method 600 for implementing a virtual anchor. The example method 600 may be performed by elements of a control device (e.g., control device 103), such as by one or more processors (e.g., processor 105) or associated system, or by other suitable elements, modules, systems, or combinations thereof. For instance, the memory may store instructions that, when executed by the one or more processors, cause control device to perform one or more of the operations in method 600. The example method may rely on the use of memory or storage elements (e.g., memory 107) during processing or analysis, or for data retention or storage.

As discussed above, when a tool is passed through the working channel of the endoscope, challenges in steering (e.g., articulating the steerable tip) arise due to the increasing stiffness of steerable tip as the tool passes through the working channel. Accordingly, the virtual anchor technology described herein provides solutions to maintaining a constant or consistent view even despite these challenges. In some cases, however, the deflection or steering of the steerable tip may be too great to allow for the tool to pass through the working channel. In such examples, the distal tip may need to be relaxed or released prior to the tool being passed through the working channel. The method 600 described herein allows for a virtual anchor position to be set prior to receiving the tool and resumed after the tool has been passed through the working channel.

At operation 602, a target is set for virtual anchoring while the distal tip is steered to a desired position. For instance, setting the target for the virtual anchor may include operations such as operations 502-510 of method 500 described above. As an example, the distal tip may be steered to a desired position and the virtual anchor option may be selected.

At operation 604, the tip steering is released or relaxed. For instance, as discussed above, steering of the tip may controlled through pull wires. When the tip steering is released in such examples, those pull wires may be returned to or towards their neutral state, which causes the steerable tip to return towards its neutral (e.g., straight position). The tip may also be more flexible in such a position.

At operation 606, the tool is received through the working channel of the endoscope while the tip steering is released. At operation 608, during the time while the steering of the tip is relaxed and while the tool is being passed through the working channel of the endoscope, the change in position of the steerable tip is tracked or monitored. The change in position may be tracked through measurements of sensors of the endoscope, such as measurements from the IMU in the steerable tip. Changes in the images captured by the camera may also be tracked or monitored to determine the change in position.

At operation 610, after the tool has passed through the working channel of the endoscope, the steerable tip is steered to is prior position according to the anchored position set in operation 602. For instance, the steerable tip is steered back to a position where the target is returned to view of the camera of the endoscope. Steering the tip to its prior anchored position may be based on the tracked position changes and/or monitored sensor measurements in operation 608. For instance, the measurements from the IMU indicate a direction and magnitude of a position change. The steering of the tip may then be performed to reverse those position changes to return the steerable tip to substantially the prior anchored position.

At operation 612, after the steerable tip has been returned to its anchored position, the steerable tip of the endoscope is virtually anchored in the anchored position. Virtually anchoring the steerable tip may include operations such as operations 512-524 of method 500 discussed above.

Those skilled in the art will recognize that the methods and systems of the present disclosure may be implemented in many manners and as such are not to be limited by the foregoing aspects and examples. In other words, functional elements being performed by a single or multiple components. In this regard, any number of the features of the different aspects described herein may be combined into single or multiple aspects, and alternate aspects having fewer than or more than all of the features herein described are possible. Functionality may also be, in whole or in part, distributed among multiple components, in manners now known or to become known.

Further, as used herein and in the claims, the phrase “at least one of element A, element B, or element C” is intended to convey any of: element A, element B, element C, elements A and B, elements A and C, elements B and C, and elements A, B, and C. In addition, one having skill in the art will understand the degree to which terms such as “about” or “substantially” convey in light of the measurement techniques utilized herein. To the extent such terms may not be clearly defined or understood by one having skill in the art, the term “about” shall mean plus or minus ten percent.

Numerous other changes may be made which will readily suggest themselves to those skilled in the art and which are encompassed in the spirit of the disclosure and as defined in the appended claims. While various aspects have been described for purposes of this disclosure, various changes and modifications may be made which are well within the scope of the disclosure. Numerous other changes may be made which will readily suggest themselves to those skilled in the art and which are encompassed in the spirit of the disclosure and as defined in the claims.

What is claimed is:

1. A method, performed by an endoscope controller, for controlling an endoscope, the method comprising:
receiving an image frame from a camera on a distal tip of an endoscope;
receiving an indication to activate a virtual anchor;
based on receiving the indication to activate the virtual anchor, setting the image frame as a reference image frame;
identifying a landmark feature in the reference image frame;
determining a first position of the landmark feature in the reference image frame;
receiving an updated image frame from the camera;
identifying the landmark feature in the updated image frame;
determining a second position of the landmark feature in the updated image frame; and
based on a difference between the first position and the second position, performing at least one of:
generating corrective steering signals to steer the distal tip; or
shifting a cropped region of the updated image frame.

2. The method of claim 1, wherein the indication to activate the virtual anchor is a manual input received by the endoscope controller.

3. The method of claim 1, further comprising displaying the reference image frame on a display of the endoscope controller, wherein identifying the landmark feature is based on manual input received via touch input received via the display.

4. The method of claim 1, further comprising:
determining a secondary characteristic for the landmark feature in the reference image frame; and
determining the secondary characteristic of the landmark feature in the updated image frame, wherein determining the positions of the landmark feature is based on the secondary characteristic.

5. The method of claim 4, wherein the secondary characteristic is at least one of a centroid, a border, an area, a major axis, or a minor axis.

6. The method of claim 1, wherein a first cropped region is set for the reference frame and shifting the cropped region of the updated image frame comprises shifting the first cropped region to a second cropped region that is in a different position than the first cropped region.

7. The method of claim 6, wherein the landmark feature is identified in the updated image frame in an area outside of the first cropped region.

8. An endoscopic imaging system comprising:
an endoscope having a steerable distal tip including a camera; and
an endoscope controller connected to the endoscope, the endoscope controller comprising a processor and memory, storing instructions that when executed by the processor, cause the endoscopic controller to perform operations comprising:
receive an indication to activate a virtual anchor;
based on receiving the indication to activate the virtual anchor, set a current image frame from the camera as a reference image frame;
identify a first position of a landmark feature in the reference image frame;
receive an updated image frame captured subsequent to the reference image frame;
identify a second position of the landmark feature in the updated image frame; and
based on a difference between the first position and the second position, perform at least one of:
generate corrective steering signals to steer the distal tip; or
shift a cropped region of the updated image frame.

9. The endoscopic imaging system of claim 8, wherein the endoscopic controller is a video laryngoscope.

10. The endoscopic imaging system of claim 8, wherein the corrective steering signals are generated to steer the distal tip.

11. The endoscopic imaging system of claim 8, wherein the cropped region of the updated image frame is shifted.

12. The endoscopic imaging system of claim 8, wherein the endoscope further comprises:
a collapsible working channel, positioned on an outer surface of the endoscope, capable of selectively collapsing and expanding to receive a surgical tool, the collapsible working channel comprising:
a proximal opening; and
a distal opening positioned proximally from steerable distal tip.

13. An endoscope comprising:
a proximal end;
a steerable distal tip including an articulating segment; and
a collapsible working channel, positioned on an outer surface of the endoscope, capable of selectively collapsing and expanding to receive a surgical tool, the collapsible working channel comprising:
a proximal opening; and
a distal opening positioned proximally from the articulating segment.

14. The endoscope of claim 13, wherein the distal opening is offset from the articulating segment by a distance between 1-5 cm.

15. The endoscope of claim 13, wherein the articulating segment is a first articulating segment, and the endoscope further comprises a second articulating segment adjacent the first articulating segment, wherein the distal opening is positioned proximally from both the first and second articulating segments.
